(19) United States
(12) Patent Application Publication
(10) Pub. No.: US 2024/0039190 A1
(43) Pub. Date: Feb. 1, 2024

(54) WEARABLE NEUROSTIMULATOR WITH RIVET CONNECTION

(71) Applicant: AVATION MEDICAL, INC., Columbus, OH (US)

(72) Inventors: Austin Morgan, Westerville, OH (US); David DeMuth, Westerville, OH (US); Manish Vaishya, Valencia, CA (US)

(21) Appl. No.: 18/361,611

(22) Filed: Jul. 28, 2023

Related U.S. Application Data
(60) Provisional application No. 63/394,021, filed on Aug. 1, 2022.

(51) Int. Cl. H01R 12/62; H01R 4/06; H05K 1/11; H05K 1/14; A61N 1/04

(52) U.S. Cl. CPC H01R 12/62; H01R 4/06; H05K 1/116; H05K 1/144; A61N 1/0484; A61N 1/0456; H05K 2201/042; H01R 4/48

(57) ABSTRACT

A wearable neurostimulator includes a fabric structure, a flexible circuit printed on the fabric structure with exposed electrically conductive portions, and a rigid PCB including an electrical circuit and exposed electrically conductive portions. A rivet includes a shank that extends through the fabric structure, conductive pads, PCB, and conductive vias. The rivet electrically connects the conductive portions of the PCB circuit to the conductive portions of the flexible circuit.

### RELATED APPLICATION

This application claims the benefit of U.S. Provisional Application Ser. No. 63/394,021, filed on Aug. 1, 2022, the disclosure of which is hereby incorporated by reference in its entirety.

### BACKGROUND

Wearable neurostimulators (“wearables,” as used herein) are stimulator devices that the user can wear during application of stimulation therapy. Wearables include a fabric structure that supports stimulation contacts and maintains their positions on the user, in contact with the user's skin. As such, wearables can come in a variety of forms, such as sleeves, braces (ankle, foot, knee, elbow, hand/wrist, back, neck, shoulder, etc.), garments (shirts, shorts, leggings, etc.). Wearables often use screen printed electrically conductive inks, such as carbon and silver inks, that are heat applied onto fabrics to create flexible electronic components. Electrical and mechanical integration between the flexible electronics and traditional, rigid printed circuit boards (PCB) and PCB-mounted electronics, which drive the stimulator system, can be difficult to manufacture in an efficient, repeatable, and reliable manner.

Traditionally, there are two common methods for connecting flexible electronics to rigid electronics: 1) conductive epoxy or 2) crimped pin connectors. Conductive epoxy is difficult to deposit, requires a curing step, lacks mechanical strength, and can be difficult to scale up to mass production. Crimped pins are bulky, require a semi-rigid backing, and require specific geometry that can be difficult to maintain when connecting flexible and/or stretchable components, such as printed fabric circuit materials.

### SUMMARY

A wearable neurostimulator includes a fabric structure, a flexible circuit printed on the fabric structure with exposed electrically conductive portions, and a rigid PCB including an electrical circuit and exposed electrically conductive portions. A rivet includes a shank that extends through the fabric structure, conductive pads, PCB, and conductive vias. The rivet electrically connects the conductive portions of the PCB circuit to the conductive portions of the flexible circuit.

According to one aspect, the PCB and the fabric structure can be positioned so that the conductive portions of the flexible circuit and PCB circuit are positioned in engagement with each other. The rivet can apply a clamping force to the PCB and the Fabric structure that maintains their contact.

According to another aspect, the rivet can be is constructed of an electrically conductive material and helps conduct electricity from the conductive portions of the flexible circuit to the conductive portions of the PCB circuit.

According to another aspect, the rivet can be a double sided rivet comprising a central conductive flange positioned between the PCB and the fabric structure. The flange can engage the conductive portions on the PCB circuit and the conductive portions on the flexible circuit.

According to another aspect, the wearable neurostimulator can include a conductive spacer fitted onto the rivet shank between the PCB and the fabric structure. The spacer can engage the conductive portions of the flexible circuit and the conductive portions of the PCB circuit.

According to another aspect, the spacer can be a waved spacer including a series of radially extending waves that engage the conductive portions of the flexible circuit and the conductive portions of the PCB circuit. The waves can be configured to deform when the rivet is applied. The waves can urge a resilient compressive force on the adjacent portions of the fabric and the PCB to help maintain electrical contact between the conductive portions of the flexible circuit and the conductive portions of the PCB circuit.

According to another aspect, the wearable neurostimulator can also include a housing for supporting the PCB and the fabric structure relative to each other and a pogo assembly. The pogo assembly can include a pogo housing mounted on the PCB in electrical contact with the conductive portions of the PCB circuit. The pogo assembly can also include a pogo pin mounted in the pogo housing and spring biased out of the housing. The rivet can extend through the fabric structure and the flexible circuit and can be electrically connected to the flexible circuit. The rivet can include a head presented toward the pogo pin. The pogo pin can be spring biased into engagement with the rivet head and can maintain electrical contact between the PCB circuit and the flexible circuit on the fabric structure, while permitting some relative movement between the fabric structure and the PCB during use.

According to another aspect, the conductive portion of the PCB circuit can include one or more conductive vias exposed on a sidewall of a through hole of the PCB through which the rivet shank extends.

According to another aspect, the conductive portion of the PCB circuit can include a plated through hole of the PCB through which the rivet shank extends.

According to another aspect, the conductive portion of the PCB circuit can include a pad surrounding a through hole of the PCB through which the rivet shank extends.

According to another aspect, the PCB can include a through hole through which the rivet shank extends. The PCB circuit can include a pad surrounding the through hole on an upper surface of the PCB, a pad surrounding the through hole on a lower surface of the PCB, and a side wall of the through hole that is plated with a conductive material and that is electrically continuous with the pads on both the upper and lower surfaces of the PCB.

### DRAWINGS

FIG. 1 is a schematic illustration of a wearable neurostimulator with a rivet connection, according to a first example configuration.

FIG. 2 is a schematic illustration of a wearable neurostimulator with a rivet connection, according to a second example configuration.

FIG. 3 is a schematic illustration of a wearable neurostimulator with a rivet connection, according to a third example configuration.

FIG. 4 is a schematic illustration of a wearable neurostimulator with a rivet connection, according to a fourth example configuration.

FIG. 5 is a schematic illustration of a wearable neurostimulator with a rivet connection, according to a fifth example configuration.

FIG. 6 is a schematic illustration of a wearable neurostimulator with a rivet connection, according to a sixth example configuration.

FIG. 7A is an exploded schematic illustration depicting the assembly of a wearable neurostimulator with a rivet connection.

FIG. 7B is a sectional view taken generally along line 7B-7B in FIG. 7A.

FIG. 7C is a sectional view taken generally along line 7C-7C in FIG. 7A.

FIG. 7D is a sectional view taken generally along line 7D-7D in FIG. 7A.

### DESCRIPTION

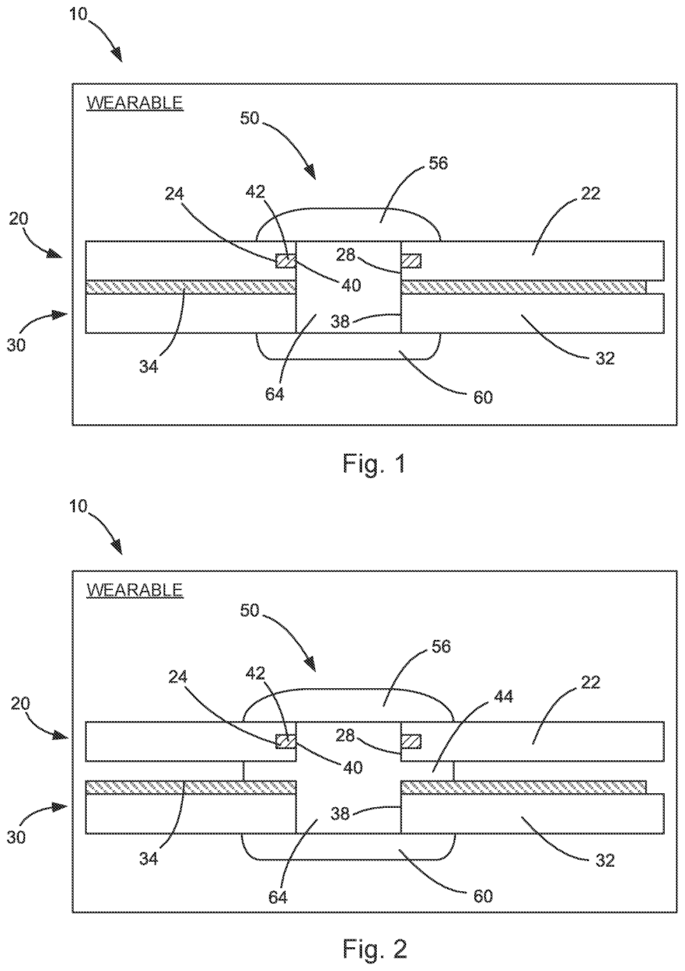

A method for achieving electrical and mechanical connection between flexible electronics, such as printed fabric circuits, and rigid printed circuit boards (PCBs) utilizes conductive, i.e., metal or metallic, rivets that extend through and form a compressed connection between the fabric, conductive ink, and PCB. The rivet connection(s) hold the conductive ink in intimate contact with electrically conductive elements on the PCB. Example configurations of wearables 10 that incorporate this configuration are depicted in FIGS. 1-6.

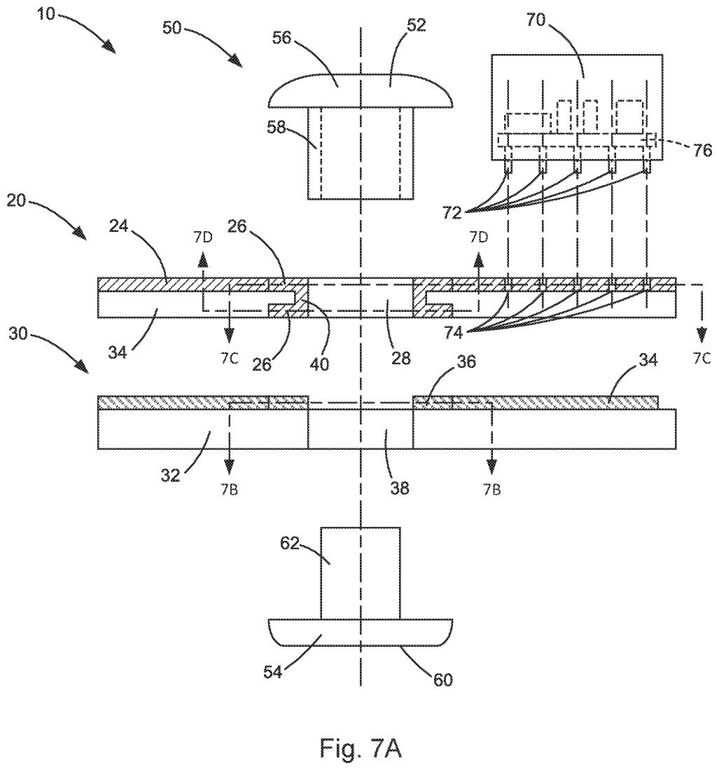

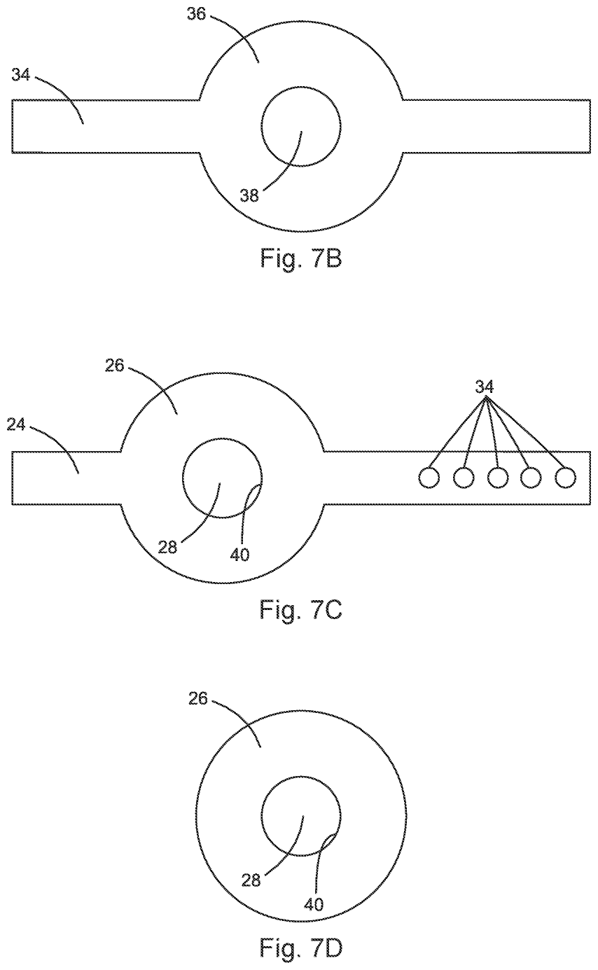

An example assembly of the wearable configurations is illustrated in FIGS. 7A-7D. In this example assembly, as is the case with each of the example configurations of FIGS. 1-6, a rivet connection relies on the rigidity of the PCB 20 and the rivet 50 to generate mechanical strength without requiring any additional rigid components, such as crimped pins. Rivet materials are much stronger than acrylic based epoxies resulting in higher tensile strength between the PCB 20 and flexible electronic component 30, which include flexible circuits 34 formed by one or more layers of conductive ink printed on a fabric sheet 32 of the wearable 10. The rivets 50 are also smaller than crimped pins and can allow for integration with the PCB 20 along the entirety of the PCB rather than via a header along the edge, which are required to accept crimped pin connections. Pressing rivets takes seconds whereas curing epoxy takes a considerable amount of time, sometimes hours. Rivet pressing can easily be automated with pneumatic feeding systems and presses.

The wearable fabric 32 can be any type of fabric, woven or non-woven, formed of organic materials, synthetic materials, or organic/synthetic blends. Neoprene is one such material that can form the wearable fabric 32 portion of the wearable 10. The flexible circuits 34 can include landing pads 36 formed by exposed conductive material, i.e., printed carbon and silver inks, on the surface of the fabric 32. A small hole 38 is punched through the fabric 32 and electrically conductive ink of the pad 36.

An electronics module 70 can include or be mounted on the rigid PCB 20, for example, via pins 72 that are received in holes 74 in the PCB. The pins 62 can be secured by known means, such as solder connections or compliant pin connections. The electronics module 70 can, for example, include electronic stimulator components. In some configurations, the electronics module 70 can include the PCB 20. In other configurations, the electronics module 70 can be mounted on the PCB 20. In this latter alternative, the PCB 20 can be separate from a PCB 76 of the electronics module, so that the PCB 20 of the wearable makes an electrical connection to the PCB 76 of the stimulator electronics module 70.

The PCB 20 includes a rigid substrate 22 upon which printed circuits 24 are supported. The printed circuits 24 include pads 26 that surround a through hole 28 for receiving the rivet 50. The through hole 28 can be plated through and can thus include a plated sidewall 40 for engaging and providing an electrical connection to the rivet 50. An additional pad 26 can be provided on the underside of the substrate 32 for making contact, both physical and electrical, with the pads 36 of the flexible circuits 34 on the flexible electronic component 30.

The rivet 50 can have any known rivet configuration. In an example configuration shown in FIG. 7, the rivet 50 includes two components: a female component 52 and a male component 54. The female component 52 includes a head 56 and a shank 58, which is hollow and cylindrical. The male component 54 includes a base 60 and a shank 62, which is solid, cylindrical, and configured to be received within the shank 58 of the female component 52. Through the application of a compressive force, the male and female components 52, 54 are joined together through material deformation, creating a shank 64 of the rivet 50, which includes both component shanks 58 and 62. In doing so, the head 56 and base 60 apply a compressive force to the PCB and the flexible electronic component 30. In the example configuration of FIG. 7, the head 56 of the female component 52 engages the pad 26 of the PCB circuit 24. The shank 64 of the rivet 50 engages the sidewall plating 40. The pad 26 on the underside of the PCB 20 can engage the pad 36 on the flexible electronic component 30. Through these engagements, electrical connectivity is achieved between the PCB circuits 24, the flexible electronic component 30, and the electronics module 70 by way of the rivet 50.

Example configurations of the wearable 10 are illustrated in FIGS. 1-5.

### Example Configuration #1

Referring to FIG. 1, in this the example configuration of the wearable 10, the PCB circuit 24 includes conductive vias 42 which form a conductive portion of the sidewall 40 of the through hole 28 in the PCB 20. In this example configuration, the portion of the PCB circuit 24 forming the vias 42 are internal to the rigid PCB substrate 22. The vias 42 could, however, be in the form of surface pads (see, e.g., FIGS. 7A-7D). The vias 42 are connected to the PCB circuit 24 (see, e.g., FIG. 7A). the PCB and the flexible electronic component 30 are compressed between the rivet head 56 and base 60. The vias 42 are exposed on the sidewall of the through hole 28. The fabric 32 of the wearable includes a flexible circuit 34 formed with printed conductive ink. Together, the fabric and ink 34 form the flexible electronic component 30. Electrical contact between the flexible circuit 34 of the flexible electronic component 30 and the vias 42 of the PCB is provided by the rivet 50. An interference between the through holes 28, 38 and the rivet shank 64 can further establish and maintain this electrical connection.

The rivet 50 includes the shank 64 that extends through the aligned openings 28, 38 in the PCB 20, the vias 42, the flexible circuit 34, and the fabric 32. The rivet 50 includes a base 60 that is pre-formed and includes a flat that engages the fabric. With the shank 64 installed through the overlying structures as shown in FIG. 1, a mechanical tool, such as a die or press tool, is used to deform the rivet material to form the head 56, which creates an assembly configuration that locks the stacked components together. In applying the rivet 50, a residual compression force can be applied to partially deform the fabric 32 so that a tight fit and electrical continuity are maintained.

### Example Configuration #2

Referring to FIG. 2, an example configuration of the wearable 10 is similar to FIG. 1, the exception being that the rivet 50 is a double-sided rivet that includes a center flange 44. In this instance, the flange 44 is sandwiched between the PCB 20 and the flexible circuit 34 on the fabric 32. In this example configuration, the portion of the PCB circuit 24 forming the vias 42 are internal to the rigid PCB substrate 22. The vias 42 could, however, be in the form of surface pads (see, e.g., FIGS. 7A-7D). The shank 64 of the rivet 50 contacts the inner surface 40 of the vias 42 of the PCB circuit 24. The flange 44 engages and makes electrical contact with the flexible circuit 34. Therefore, the rivet provides electrically conductive path between the vias 42 of the PCB 20 and the flexible circuit 34 of the flexible electronic component 30.

The components are assembled on the shank 64 on both sides of the flange 44. Due to the center location of the flange 44, neither the base 60 nor the head 56 are pre-formed structures. Instead, once the components are assembled on the shank 64 on opposite sides of the flange 44, mechanical tools, such as a dies or press tools, are used to deform the rivet material to form both the head 56 and the base 60. In this configuration, the rivet 50 can be formed of a single piece of material. In applying the rivet 50, a residual compression force can be applied to partially deform the fabric 32 so that a tight fit and electrical continuity between the components are maintained.

### Example Configuration #3

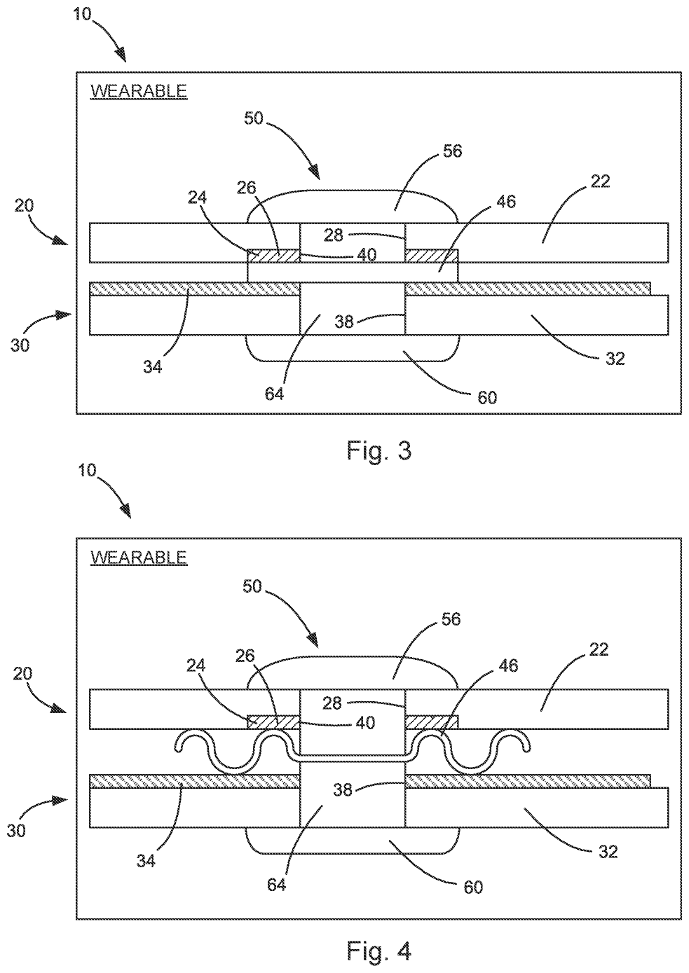

Referring to FIG. 3, an example configuration of the wearable 10 is similar to FIGS. 1 and 2, the exception being that the rivet 50 includes a spacer 46 made from electrically conductive material and applied onto the rivet shank 64. The rivet 50 is therefore similar or identical to FIG. 1, with the spacer 46 secured to the shank resembling the configuration of FIG. 2. The spacer 46 engages and makes electrical contact with the flexible circuit 34 of the flexible electronic component 30 and the pad 26 of the PCB circuit 24. Therefore, the rivet provides electrically conductive path between the circuit 24 of the PCB 20 and the flexible circuit 34 of the flexible electronic component 30.

The components, i.e., PCB 20, flexible electronic component 30, and spacer 46, are assembled on the shank 64 against the base 60 in the order shown in FIG. 3. Once the components are assembled, mechanical tools, such as a dies or press tools, are used to deform the rivet material to form the head 56. This creates an interference that locks the stacked components together. In applying the rivet 50, a residual compression force can be applied to partially deform the fabric 32 so that a tight fit and electric continuity are maintained.

### Example Configuration #4

Referring to FIG. 4, an example configuration of the wearable 10 is similar to FIG. 3, the exception being that the spacer 46 is waved, as opposed to flat. The assemblage of the rivet 50, with the flexible electronic component 30, spacer 46, and PCB 20 stacked as shown, is compressed during the formation of the rivet head 56. As a result, the waved structure of the spacer 46 is deformed. The resilience of the material (e.g., metal) used to form the waved spacer 46 causes the spacer to exert an outward (up and down, as shown) force that maintains engagement of the conductive pads 26 of the PCB circuit 24 and the flexible circuit 34 on the fabric 32. In this manner, the electrical connection between the flexible electronic component 30 and the PCB 20 is enhanced and maintained.

### Example Configuration #5

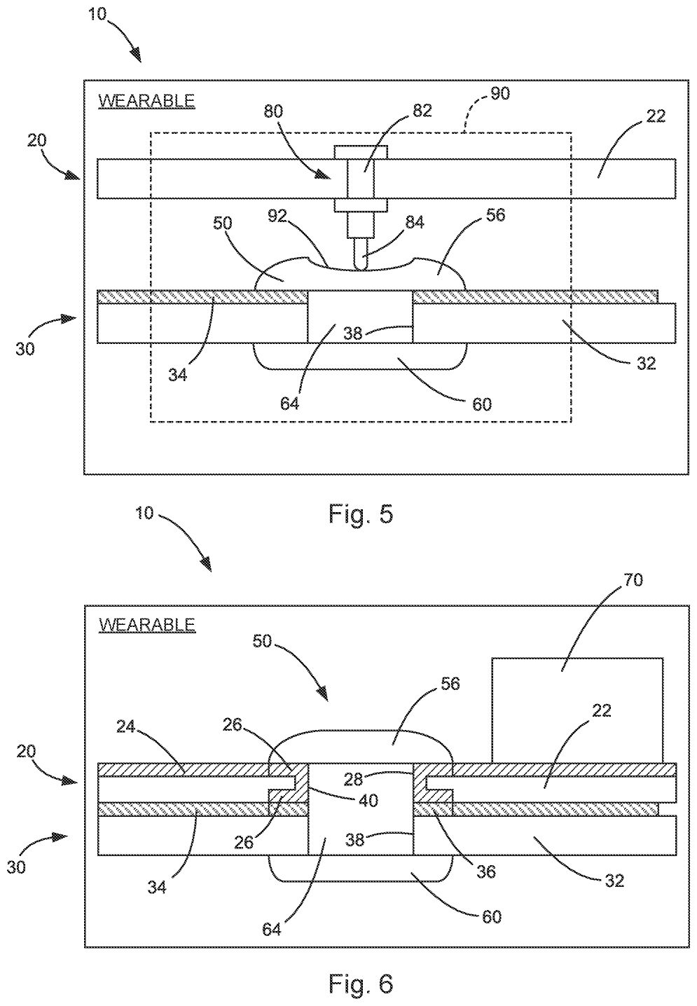

Referring to FIG. 5, an example configuration of the wearable 10 utilizes a pogo assembly 80 to maintain electrical contact between the PCB circuit 24 and the flexible circuit 34 of the flexible electronic component 30. In this configuration, the PCB and the flexible electronic component 30, or portions thereof, are supported by a housing 90 that maintains their relative positions, as shown in FIG. 5. The rivet 50 extends through the fabric 32 and the flexible circuit 34 and is therefore electrically connected to the flexible electronic component 30. The PCB 20 is supported by the housing 90 spaced from the flexible electronic component 30 (above the flexible circuit, as shown in FIG. 5).

The pogo assembly 80 in includes a pogo spring housing 82 that extends through and is connected to the PCB 20, both mechanically, to the board 22 itself, and electrically, to the PCB circuit 24. A pogo pin 84 is supported by the pogo housing 82 and is spring biased out of the housing (downward, as shown in FIG. 5). The rivet head 56 a recess 92 configured to receive the pogo pin 84, which establishes an electrical connection between the flexible circuit 34 and the PCB circuit 24.

The pogo assembly 80 allows for some relative movement between the fabric 32 and the rigid PCB 20. As their relative positions shift, the pogo pin 84 can slide laterally in the recess 92. Up/down relative movement between the flexible electronic component 30 and the PCB 20 can be taken up by telescopic movement of the pogo pin 84 into and out of the pogo housing 82, under the bias of the spring (not shown) housed therein. Because of this, electrical contact can be maintained even if there is some shifting between the PCB 20 and the flexible electronic component 30, such as that caused be user movements during use.

### Example Configuration #6

Referring to FIG. 6, an example configuration of the wearable 10 is similar to the configuration of FIG. 1. In this example configuration, the PCB 20 and flexible electronic component 30 are configured to improve electrical contact between the PCB circuit 24 and the flexible circuit 34. To do this, the through hole 28 of the PCB 20 has a plated through configuration and implements pads 26 on both sides of the substrate 22. The flexible circuit 34 of the flexible electronic component 30 implements pads 36 on its upper surface. The lower pads 26 of the PCB circuit 24 and the upper pads 36 of the flexible circuit 34 are configured to face and engage each other to enhance their areas of physical and electrical contact. The rivet 50, when applied, enhances this contact. Additionally, the plated surface 40 of the through hole 28 engages the shank 64 of the rivet 50, and the rivet head 56 engages the upper pad 26 of the PCB 20. These engagements improve the electrical contact between the PCB circuit 20 and the flexible electronic component 30.

From the above description of the invention, those skilled in the art will perceive improvements, changes and modifications. Such improvements, changes and modifications within the skill of the art are intended to be covered by the appended claims.

1. A wearable neurostimulator comprising:
a fabric structure;
a flexible circuit printed on the fabric structure with exposed electrically conductive portions;
a rigid printed circuit board (PCB) comprising a PCB circuit and exposed electrically conductive portions;
a rivet comprising a shank that extends through the fabric structure, PCB, conductive portions of the flexible circuit, and conductive portions of the PCB circuit, wherein the rivet electrically connects the conductive portions of the flexible circuit to the conductive portions of the PCB circuit.

2. The wearable neurostimulator recited in claim 1, wherein the PCB and the fabric structure are positioned so that the conductive portions of the flexible circuit and PCB circuit are positioned in engagement with each other, the rivet applying a clamping force to the PCB and the Fabric structure that maintains their contact.

3. The wearable neurostimulator recited in claim 1, wherein the rivet is constructed of an electrically conductive material and helps conduct electricity from the conductive portions of the flexible circuit to the conductive portions of the PCB circuit.

4. The wearable neurostimulator recited in claim 1, wherein the rivet is a double sided rivet comprising a central conductive flange positioned between the PCB and the fabric structure, the flange engaging the conductive portions on the PCB circuit and the conductive portions on the flexible circuit.

5. The wearable neurostimulator recited in claim 1, further comprising a conductive spacer fitted onto the rivet shank between the PCB and the fabric structure, the spacer engaging the conductive portions of the flexible circuit and the conductive portions of the PCB circuit.

6. The wearable neurostimulator recited in claim 5, wherein the spacer comprises a waved spacer comprising a series of radially extending waves that engage the conductive portions of the flexible circuit and the conductive portions of the PCB circuit, wherein the waves are configured to deform when the rivet is applied, the waves urging a resilient compressive force on the adjacent portions of the fabric and the PCB to help maintain electrical contact between the conductive portions of the flexible circuit and the conductive portions of the PCB circuit.

7. The wearable neurostimulator recited in claim 1, further comprising:
a housing for supporting the PCB and the fabric structure relative to each other;
a pogo assembly comprising a pogo housing mounted on the PCB in electrical contact with the conductive portions of the PCB circuit, the pogo assembly comprising a pogo pin mounted in the pogo housing and spring biased out of the housing;
wherein the rivet extends through the fabric structure and the flexible circuit and is electrically connected to the flexible circuit, the rivet comprising a head presented toward the pogo pin;
wherein the pogo pin is spring biased into engagement with the rivet head and maintains electrical contact between the PCB circuit and the flexible circuit on the fabric structure, while permitting some relative movement between the fabric structure and the PCB during use.

8. The wearable neurostimulator recited in claim 1, wherein the conductive portion of the PCB circuit comprises one or more conductive vias exposed on a sidewall of a through hole of the PCB through which the rivet shank extends.

9. The wearable neurostimulator recited in claim 1, wherein the conductive portion of the PCB circuit comprises a plated through hole of the PCB through which the rivet shank extends.

10. The wearable neurostimulator recited in claim 1, wherein the conductive portion of the PCB circuit comprises a pad surrounding a through hole of the PCB through which the rivet shank extends.

11. The wearable neurostimulator recited in claim 1, wherein the PCB comprises a through hole through which the rivet shank extends, wherein the PCB circuit comprises a pad surrounding the through hole on an upper surface of the PCB, a pad surrounding the through hole on a lower surface of the PCB, and a side wall of the through hole that is plated with a conductive material and that is electrically continuous with the pads on both the upper and lower surfaces of the PCB.
